(19) United States
(12) Patent Application Publication
(10) Pub. No.: US 2008/0241243 A1
(43) Pub. Date: Oct. 2, 2008

(54) Drug delivery device for providing local analgesia, local anesthesia or nerve blockade

(76) Inventors: Michael Myers, Ashburn, VA (US); Philip Wallace Reginald, Buckinghamshire (GB)

Correspondence Address: Jane Massey Licata;Licata & Tyrrell P.C., 66 E. Main Street, Marlton, NJ, 08053, US

(21) Appl. No.: 11/692,337

(22) Filed: Mar. 28, 2007

(51) Int. Cl. A61K 31/38; A61K 31/16; A61K 31/445; A61K 47/42; A61K 9/14; A61P 43/00

(52) U.S. Cl. 424/484; 514/330; 514/447; 514/626; 514/772

(57) ABSTRACT

The invention relates to a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration.

### FIELD OF THE INVENTION

The present invention relates to a drug delivery device for providing local analgesia, local anesthesia or nerve blockade and a method for providing local analgesia, local anesthesia or nerve blockade in a human or animal in need thereof.

### RELATED BACKGROUND ART

Post-surgical pain is a complex response to tissue trauma during surgery that stimulates hypersensitivity of the central nervous system. Post-operative pain increases the possibility of post-surgical complications, raises the cost of medical care and, most importantly, interferes with recovery and return to normal activities of daily living. Management of post-surgical pain is a basic patient right. When pain is controlled or removed, a patient is better able to participate in activities such as walking or eating, which will encourage his or her recovery. Patients will also sleep better, which aids the healing process.

Collagen sponges have been used globally as hemostatic agents. The present inventors have developed a drug delivery device in the optional form of a collagen sponge impregnated with at least one anesthetic such as bupivacaine hydrochloride, intended for use in the management of post-operative pain following surgery including, but not limited to, moderate/major orthopedic, abdominal, gynecological or thoracic surgery. The at least one anesthetic is contained, in one embodiment, within a collagen matrix comprised of fibrillar collagen, such as Type 1 collagen purified from bovine Achilles tendons.

Bupivacaine, introduced in 1963, is a widely used amide local anesthetic with a prolonged duration of action. It affects sensory nerves more than motor nerves and can also be used to provide several days' effective analgesia without motor blockade.

Bupivacaine is characterized by its longer duration and slow onset compared with other local anesthetics. Bupivacaine is markedly cardiotoxic. Systemic exposure to excessive quantities of bupivacaine mainly result in central nervous system (CNS) and cardiovascular effects—CNS effects usually occur at lower blood plasma concentrations and additional cardiovascular effects present at higher concentrations, though cardiovascular collapse may also occur with low concentrations. CNS effects may include CNS excitation (nervousness, tingling around the mouth, tinnitus, tremor, dizziness, blurred vision, seizures) followed by depression (drowsiness, loss of consciousness, respiratory depression and apnea). Cardiovascular effects include hypotension, bradycardia, arrhythmias, and/or cardiac arrest—some of which may be due to hypoxemia secondary to respiratory depression.

### Incisional Local Anesthesia

Wound infiltration with local anesthesia is used widely for postoperative pain: it is simple, safe and low cost. However, it is unclear whether differences in surgical procedure or whether visceral components influence efficacy. Incisional anesthesia includes infiltration, topical administration or instillation of local anesthetic at the following sites: skin, subcutaneous tissue, fascia, muscle and/or the parietal peritoneum. However, in spite of widespread use, wound infiltration is still inconsistently and randomly used by many surgeons and anesthetists.

Although there is a great number of papers and reviews on this topic, there is little consensus available on when and after which surgical procedures, incisional local anesthesia may provide clinically relevant post-operative pain alleviation. Of special interest may be to what extent differences in surgical procedure or involvement of visceral components influence efficacy. Incisional local anesthesia has been studied in a broad range of surgical models, including abdominal hysterectomy, inguinal herniotomy, open cholecystectomy, appendectomy, Caesarean section and other laparotomy procedures. A laparotomy is a surgical maneuver involving an incision through the abdominal wall to gain access into the abdominal cavity.

The anesthetics assessed for post-operative pain relief include lidocaine, bupivacaine, ropivacaine and mepivacaine, which all belong to the amino amide anesthetic group. On review of the data on the use of incisional anesthesia in hysterectomy surgery, conflicting results have been obtained. In a study by Sinclair et al, 1996, 500 mg of lidocaine administered as an aerosol subcutaneously caused a significant reduction of approximately 50% in pain scores and supplementary analgesic consumption during the first 24 hours of the study, but not later. In a study by Hannibal and co-workers, 0.25% bupivacaine solution 45 ml infiltrated subfascially and subcutaneously caused a 50% reduction in analgesic consumption but not in pain scores or time to first analgesic request. In contrast, two studies evaluating subcutaneous infiltration of bupivacaine solution compared with no treatment showed no improvement in analgesia (Cobby et al, 1997, Victory et al, 1995).

Studies in other models have shown short-term analgesic effects over 4 to 7 hours. In three studies on Caesarean section, 0.25% or 0.5% bupivacaine 20 ml caused a 20-50% reduction in analgesic consumption but this effect only lasted for 4 hours. In another study in upper abdominal surgery, only a slight reduction in daily morphine administration (supplemental intramuscular morphine) (10 mg) was noted and a reduction in visual analogue scoring (VAS) only during mobilization was recorded (50 mm) (Bartholdy et al, 1994). In a review of incisional anesthesia for the control of post-operative pain, Møiniche et al (1998) assessed 26 studies involving over 1200 patients in surgeries using abdominal incision. The results showed a consistent statistical and clinical effect of incisional anesthesia in herniotomy surgery, although the analgesia was short-lived (2-7 hours). However, in the other surgical models evaluated including hysterectomy the results were variable between studies.

Of the 26 studies evaluated (Møiniche et al, 1998), eight were unequivocally negative. Although the majority of studies showed significant differences in at least one pain measure, several were of questionable clinical importance and the authors were surprised that local anesthesia was not associated with more consistent positive results. The authors also noted the importance in the technique used and site to administer the anesthetics.

Since the 1998 review, surgical wound infiltration trials have continued to be performed and published as the practice remains relatively common despite the lack of strong evidence. For example, a group in Leicester, UK, have published two hysterectomy trials (Klein et al, 2000 and Ng et al, 2002), which at best have concluded a duration of effect only up to 4 hours post-operatively.

In contrast, trials where bupivacaine has been instilled post-operatively on a continuous or intermittent basis via an indwelling catheter have tended to prove much more successful and effective. Gupta et al (2004) compared an infusion of normal saline against an infusion of 0.25% levobupivacaine (12.5 mg/hr) over 24 hours and showed a significant reduction in incisional pain, deep pain and pain on coughing at 1-2 hours post hysterectomy surgery. Total ketobemidone (PCA narcotic) was significantly reduced over the 4-24 hour period and the authors conclude that the intraperitoneal infusion of levobupivacaine has significant opioid sparing effects after elective abdominal hysterectomy.

The apparent efficacy of anesthetic infusions explains the widespread use of ambulatory pain pumps, such as I-Flow's ON-Q® Painbuster. However, such continuous infusion devices use much higher total doses of bupivacaine (between 2.5 mg/hr and 50 mg/hr with a maximum dosing duration of 5 and 2 days, respectively) and of course are less convenient than a biodegradable implant. The in-dwelling catheter used in the pain pump system can lead to infection and must be removed by a physician or nurse. In contrast, a drug delivery device such as the bupivacaine-collagen sponge provides, as will be demonstrated hereunder, effective, long lasting analgesia but at a dose only equivalent to a once-off bolus infiltration of the wound.

In the present invention, this long lasting analgesia is achieved through the use of a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration. Accordingly, the invention provides, in a first aspect, a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration.

It is hypothesized that such drug delivery devices as the bupivacaine-collagen sponge will afford post-operative pain management to patients without adverse effects associated with toxicity from the collagen sponge or elevated systemic anesthetic (such as bupivacaine) levels. It is hypothesized that such drug delivery devices as the bupivacaine-collagen sponge will provide local pain relief to patients for up to 48 or 72 hours at the surgical site and reduce the patient's demand for systemic analgesia and the associated adverse effects.

### SUMMARY OF THE INVENTION

The present invention is directed to a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration. The present invention is directed, in an optional embodiment, to a biodegradable, leave-behind device.

### BRIEF DESCRIPTION OF THE FIGURES

FIG. 1 shows, schematically, a flow diagram for the production of collagen.

FIG. 2 shows, schematically, a flow diagram for the production of a drug delivery device in the optional form of a bupivacaine-collagen sponge.

### DETAILED DESCRIPTION OF THE INVENTION

This invention relates to a drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof the at least one drug substance being substantially homogeneously dispersed in the collagen matrix, and the at least one drug substance being present in an amount sufficient to provide a duration of local analgesia, local anesthesia or nerve blockade which lasts for at least about one day after administration.

In a second aspect, the invention relates to a method for providing local analgesia, local anesthesia or nerve blockade in a human or animal in need thereof, the method comprising administering at a site in a human or animal in need thereof a drug delivery device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration.

Optionally, in the device or method of the invention, the at least one drug substance is present in an amount sufficient to provide a duration of local analgesia, local anesthesia or nerve blockade which lasts for at least about two days after administration.

Optionally, in the device or method of the invention, the at least one drug substance is present in an amount sufficient to provide a duration of local analgesia, local anesthesia or nerve blockade which lasts for at least about three days, further optionally at least four days, after administration.

Optionally, in the device or method of the invention, the at least one drug substance is an amino amide anesthetic selected from the group comprising Bupivacaine, Levobupivacaine, Lidocaine, Mepivacaine, Prilocaine, Ropivacaine, Articaine, Trimecaine and their salts and prodrugs. Further optionally, in the device or method of the invention, the at least one drug substance is an amino amide anesthetic selected from bupivacaine and salts and prodrugs thereof.

Optionally, in the device or method of the invention, the fibrillar collagen matrix is a Type 1 collagen matrix.

Optionally, in the device or method of the invention, the fibrillar collagen matrix is a Type 1 collagen matrix and the at least one drug substance is an amino amide anesthetic selected from bupivacaine and salts and prodrugs thereof. Further optionally, the drug delivery device comprises a plurality of collagen sponges, each collagen sponge containing about 3.6 to about 8.0 mg/cm³ type I collagen and about 2.0 to about 6.0 mg/cm³ bupivacaine hydrochloride. Still further optionally, the drug delivery device comprises a plurality of collagen sponges, each collagen sponge containing about 5.6 mg/cm³ type I collagen and about 4.0 mg/cm³ bupivacaine hydrochloride.

Optionally, in the method of the invention, the method is for providing local analgesia, local anesthesia or nerve blockade in a human following laparotomy.

Optionally, in the method of the invention, the method is for providing local analgesia, local anesthesia or nerve blockade in a human following hysterectomy.

Optionally, in the method of the invention, the drug delivery device comprises a plurality of collagen sponges and wherein one sponge is divided between areas in the surgical vault, one sponge is divided and placed across the incision in the peritoneum and one sponge is divided and placed between the sheath and skin around the incision.

### Drug Substance

Suitable drug substances comprise amino amide anesthetics and amino ester anesthetics and their salts, hydrates and prodrugs. Such drug substances include, but are not limited to, amino amides such as Bupivacaine, Levobupivacaine, Lidocaine, Mepivacaine, Prilocaine, Ropivacaine, Articaine, Trimecaine and their salts and prodrugs; and amino esters such as Benzocaine, Chloroprocaine, Cocaine, Procaine, Tetracaine and their salts and prodrugs. Bupivacaine, and its salts and prodrugs is an optional drug substance. Mixtures of amino amides are contemplated, as are mixtures of amino esters. Mixtures of amino amides and amino esters are also contemplated.

Bupivacaine Hydrochloride (HCl) is a potent anesthetic and can produce moderate to prolonged anesthesia. When compared to other available amino amide anesthetics, the relatively longer duration of action coupled with its action on sensory block, rather than motor-block, permits prolonged anesthesia for post-operative pain. Bupivacaine HCl can provide effective sensory block and analgesia for several days. Bupivacaine HCl is indicated for moderate to prolonged local anesthesia and, therefore, treatment of moderate to acute pain.

Toxicity related to bupivacaine is caused by high systemic levels and are characterized by numbness of the tongue, light-headedness, dizziness and tremors, followed by convulsions and cardiovascular disorders.

The pharmacokinetics and pharmacodynamics of bupivacaine are well understood. Bupivacaine is about 95% bound to plasma proteins. Reported half-lives are from 1.5 to 5.5 hours in adults and about 8 hours in neonates. It is metabolized in the liver and is excreted in the urine, principally as metabolites with only 5 to 6% as unchanged drug. Bupivacaine is distributed into breast milk in small quantities. It crosses the placenta but the ratio of fetal to maternal concentrations is relatively low. Bupivacaine also diffuses into the cerebrospinal fluid (CSF).

The toxic threshold for bupivacaine plasma concentrations is considered to lie in the range of 2 to 4 micrograms/mL and, in the US, the maximum single recommended dose for anhydrous bupivacaine hydrochloride is 175 mg. Measurement of bupivacaine levels in the clinical setting needs to demonstrate dosing and systemic levels within these safety parameters. Thus, when it is intended to administer bupivacaine, whether in one or several collagen sponges, it is suggested that the total dose should be no more than about 250 mg, optionally no more than about 200 mg, for anhydrous bupivacaine hydrochloride.

### Collagen

Fibrillar collagen from different sources may be used including commercially available fibrillar collagen, for example, biomedical collagen from Devro Biomedical Collagen, Australia. Currently there are five known types of fibrillar collagen; Type I, II, III, V and XI. Alternatively, collagen can be extracted from tendons or hides of different animals, including horses, cattle, sheep and pigs. The attention of the skilled reader is drawn to Gelse et al (Advanced Drug Delivery Reviews 55 (2003)1531-1546), the whole contents of which are incorporated herein by reference for further details on the various types of collagen. The present inventors have used a bovine-derived collagen Type 1 for the manufacture of bupivacaine-collagen sponges. Equine-derived collagen Type 1 is also suitable for use in the present invention, as are fibrillar collagen such as type 1 collagen from pigs and sheep. Type 1 collagen is a connective tissue extracted from animal tendons and other sources; in this case, the collagen is derived from bovine tendons. The Type 1 collagen consists of three approximately 1,050 amino-acid-long polypeptide chains, two alpha-1 chains, and one alpha-2 chain. These are coiled to form a right-hand helix (known as a triple helix) around a common axis. The rod-shaped molecule has a length of 2900 Angstrom, a diameter of 14 Angstrom and a molecular weight of approx. 300,000 Daltons.

### Method of Manufacture

The following general method of manufacture refers to type I collagen being produced from bovine tendons. However, alternative sources of fibrillar collagen such as alternative sources of type I or III collagen may be used in place of this methodology, without departing from the scope of the teaching of this invention.

The following general method of manufacture refers to bupivacaine as the drug substance. It will be appreciated that alternative drug substance(s), or additional drug substance(s) (i.e. additional to bupivacaine), may be used in place of bupivacaine alone, without departing from the scope of the teaching of this invention.

### Collagen

Production of Type 1 Collagen from Bovine Tendons The collagen is extracted from bovine Achilles tendon. During the manufacturing process, bovine tendons are first treated with 1N sodium hydroxide (NaOH) to clean and purify the material and to deplete the fat content followed by neutralization with 1N HCl. This step is followed by treatment with 0.9% sodium chloride (NaCl) solution to remove low molecular weight soluble components of the collagen. A treatment with hydrogen peroxide solution ensures bleaching of the tendons.

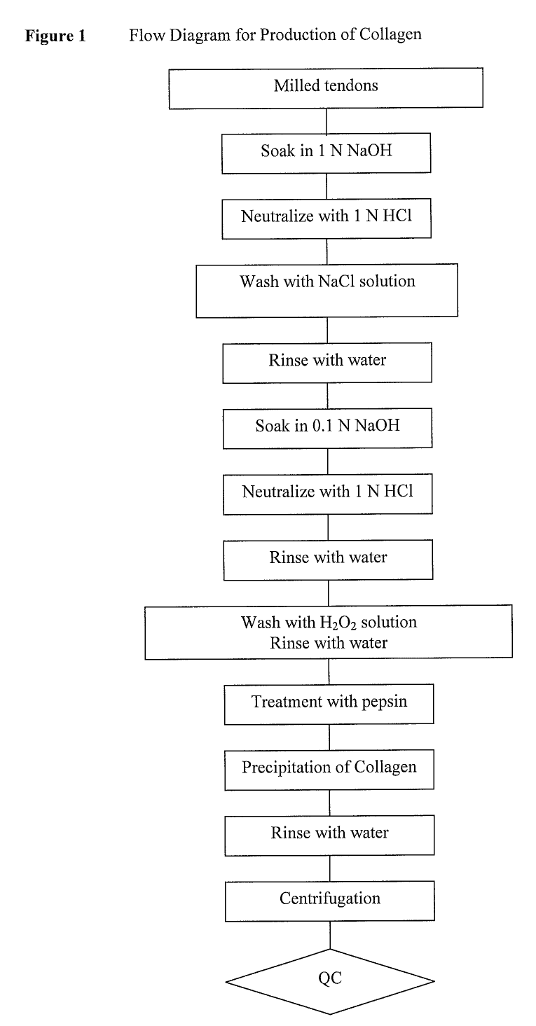

Reduction of the particle size of the collagen material is followed by fermentative breakdown using pepsin. Treatment with pepsin is used to degrade contaminating serum protein components, primarily bovine serum albumin and causes the detachment of non-helical portions of the collagen molecule (telopeptides). After filtration, precipitation of the collagen is accomplished by means of manipulation of the pH (from acidic pH to neutral pH). The fibrillar Type 1 collagen material is finally precipitated out of solution, washed again with distilled water to remove residual pepsin and then concentrated by means of centrifugation. The production process is outlined in FIG. 1.

### Bupivacaine Collagen Sponge—Method of Manufacture

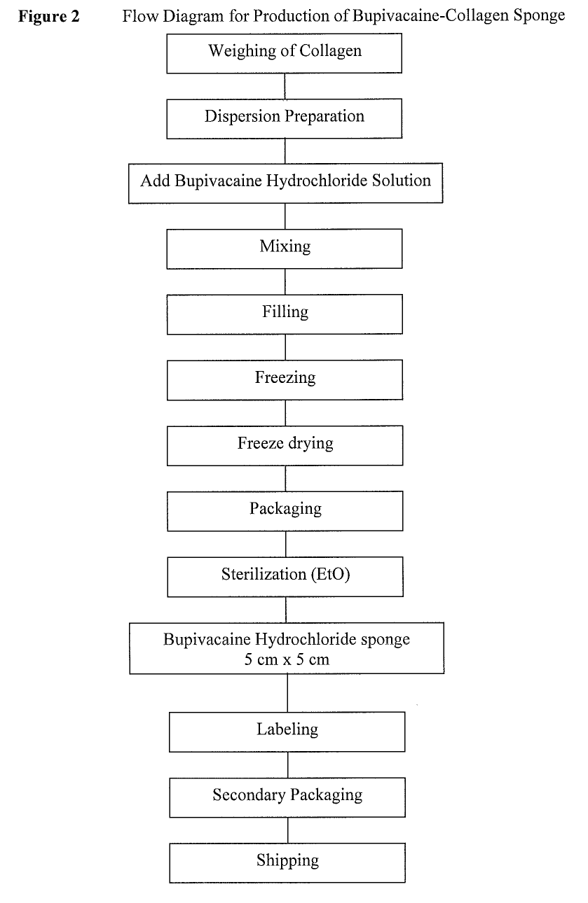

FIG. 2 is a flow diagram representing the production of the bupivacaine-collagen sponge. The skilled reader will appreciate that other drug substance(s) may be used in place of, or in addition to, the bupivacaine.

### Compounding Process and Equipment

The fibrillar Type 1 collagen material prepared as in FIG. 1 is added to pre-heated water (below 42° C.) in a stainless steel (SS) vessel. Collagen swelling and subsequent dispersion formation is afforded by the use of a high-shear homogenizer. The homogenizer employed possesses a rotor-stator head that is designed to create high shear forces by pulling the collagen material through the rotating homogenizer head and forcing it against the proximal stationary stator head. It is this design that facilitates the high shear forces required to separate the fibrous collagen mass at the beginning of dispersion preparation.

Following completion of collagen dispersion formation, the dispersion is transferred to a closed heated jacketed vessel for final compounding. The jacket temperature is maintained at 36-38° C.

The or each drug substance (such as bupivacaine HCl) raw material is first dissolved in a portion of water at room temperature and is then introduced into the heat-jacketed SS vessel under low shear mixing to achieve homogeneity in the drug-loaded collagen dispersion. The collagen/bupivacaine dispersion is a free flowing opaque white to off-white liquid.

The dispersion is subsequently freeze-dried yielding a sponge containing, in one embodiment, 5.6 mg/cm³ of collagen and 4.0 mg/cm³ of bupivacaine HCl in a final lyophilized 5 cm×5 cm product. Other product sizes can also be manufactured including a 10 cm×10 cm sponge also containing, in an embodiment, 5.6 mg/cm³ of collagen and 4.0 mg/cm³ of bupivacaine HCl.

### Filling/Lyophilization Process and Equipment

The collagen/drug dispersion is filled into appropriately sized lyophilization molds or blister trays for freeze-drying and the filling process is performed using a positive displacement pump. The pump is valve-less, has ceramic pistons and works on the principle of positive displacement.

Upon completion of tray filling, the filled moulds or blister trays are placed into the lyophilizer. Thermocouples are placed both in product and on shelves and a conductivity probe is also employed to provide in-process feedback on process temperatures and conductivity. The lyophilization process cycle used for the bupivacaine-collagen sponge involves freezing down to a temperature of −38° C. over 3.5 hours, followed by drying to a temperature of 30° C. over 14.5 hours.

### Ethylene Oxide (EtO) Sterilization Process

The lyophilized sponge is packed into suitable packaging material, which may comprise of a sealed polyethylene blister or low density polyethylene (LDPE) sachet in an outer pouch consisting of polyethylene/LDPE laminate or aluminum foil. The product is then subjected to terminal sterilization, which can be gas-mediated ethylene oxide sterilization or radiation (gamma or electron beam).

In the preferred embodiment, sterilization by ethylene oxide gas has been selected.

Ethylene oxide (C₂H₄O) is a gas at operating temperature and sterilizes via its action as a powerful alkylating agent. Under the correct conditions, cellular constituents of organisms such as nucleic acid complexes, functional proteins and enzymes will react with ethylene oxide, causing the addition of alkyl groups. As a result of the alkylation, cell reproduction is prevented and cell death ensues. Specific processing conditions and parameters must be met to achieve this effect within the target product; including but not limited to, acceptable concentration of ethylene oxide in the chamber and a minimum water activity level within the organism. The process is essentially a chemical reaction and is therefore temperature dependent; the rate of reaction increases with temperature. The optimum temperature is within the range of 30 and 40° C. These properties define the key characteristics of the ethylene oxide sterilization process.

The process is dependent on the water content existing in the sponges and a consistent range of moisture content is achieved by equilibration of the product with atmospheric humidity prior to sterilization. An optimum water content is not less than 9%. The product is loaded into stainless steel wire mesh baskets and placed into the stainless steel sterilizer chamber using a defined loading pattern. The sterilization chamber is then evacuated to remove air and ethylene oxide is introduced until the required concentration is achieved.

Product is held under these conditions for a defined period and, on completion of the pre-determined dwell period, ethylene oxide from the chamber is exhausted to the atmosphere via catalytic converters. These units ensure catalytic conversion of ethylene oxide to carbon dioxide and water with high efficiency. The sterilization chamber and its contents are then repeatedly flushed with air to remove the remaining ethylene oxide from the chamber. After completion of post sterilization flushing, the product is transferred to a holding area for longer term aeration. This phase of the process serves to further scavenge low level residual ethylene oxide from the product and packaging. The product is held at room temperature until the limits for ethylene oxide derivative residues have been reached.

### Alternative Sterilization Process and Equipment

Radiation sterilization including gamma and electron beam may be used instead of the EtO sterilization process mentioned above.

The bupivacaine-collagen sponge manufactured under this general method of manufacture is a drug-delivery system composed of a Type 1 collagen matrix containing the amide local anesthetic bupivacaine HCl. The release of bupivacaine is primarily by dissolution and diffusion from the porous matrix with the collagen sponge acting as an inert delivery system.

### Hysterectomies

Hysterectomy is the second most common surgery among women in the United States (US). According to the National Center For Health Statistics, there were 617,000 hysterectomies performed in the US in 2004. Indications for hysterectomy include benign tumors, such as fibroids, heavy periods, painful periods and chronic pelvic pain. The most common route for performing hysterectomy is through an incision in the abdominal wall; however, about 20% are performed vaginally. Laparoscopic-assisted vaginal hysterectomy is performed when warranted.

### Pain Control in Hysterectomies

Effective postoperative pain management is important in ensuring that surgical subjects have a smooth and successful recovery after their operation. Pain after abdominal hysterectomy can be multifactorial. Incision pain, pain from deeper (visceral) structures, and particularly, dynamic pain, such as during straining, coughing, or mobilizing, can be quite severe. In one study, the authors found that visceral pain dominated during the first 48-hours after hysterectomy (Leung, 2000).

Morphine is often used via patient-controlled analgesic (PCA) pumps to control post-operative pain, but the large quantities required can lead to fatigue, nausea and vomiting, as well as the inability to mobilize because of drowsiness. Subjects usually require PCA for at least 24-hours, after which they receive oral analgesic drugs. The average postoperative narcotic consumption during the first 24-hours varies from 35 to 62 mg (Gupta, 2004) and the average postoperative morphine consumption using bupivacaine infiltration in both and superficial layers of the wound after abdominal hysterectomy was 54 mg (Klein et al, 2000) and 44 mg (Ng et al, 2002).

### Collagen Products

The properties of insoluble and soluble collagen have led to its use in a variety of medical applications ranging from heart valves to dermal implants. Soluble collagen can be used to produce biodegradable or non-biodegradable materials that give useful mechanical properties and biocompatibility.

Soluble collagen can be cross-linked to produce semi-permanent, non-absorbable implants that can be delivered by intradermal injection such as those used in facial aesthetics. These were first approved by the US Food and Drug Administration (FDA) in the 1980s.

The present collagen matrix can be a localized drug delivery system based on a fibrillar (Type I or Type III) collagen matrix, optionally derived from bovine Achilles tendons. The products are manufactured as a lyophilized sponge.

Embodiments of the invention will now be demonstrated by reference to the above-mentioned General Method of Manufacture, which is then exemplified by reference to the Clinical Study described hereunder.

Specific embodiments of the invention will now be demonstrated by reference to the following general methods of manufacture and examples. It should be understood that these examples are disclosed solely by way of illustrating the invention and should not be taken in any way to limit the scope of the present invention.

### EXAMPLES

### Clinical Study

The effective relief of pain is of paramount importance to those treating patients undergoing surgery. This should be achieved for humanitarian reasons, but there is now evidence that pain relief has significant physiological benefit. Not only does effective pain relief mean a smoother postoperative course with earlier discharge from hospital, but it may also reduce the onset of chronic pain syndromes. Topical or local administration of anesthetics directly at the surgical site has the advantage of producing high local anesthetic concentrations, while minimizing potentially toxic systemic concentrations.

The bupivacaine-collagen sponge is highly malleable and can be applied directly, rolled or folded, giving the surgeon great flexibility in terms of application in wounds scheduled for closure.

Patients received three 5 cm×5 cm (×0.5 cm thick) sponges; one sponge divided between areas in the surgical vault, one sponge divided and placed across the incision in the peritoneum and the final sponge divided and placed between the sheath and skin around the incision. Each sponge contained 50 mg of bupivacaine hydrochloride, giving a total dose of 150 mg per patient.

This was a single dose, open-label, prospective clinical study to investigate drug delivery devices in the form of a bupivacaine-collagen sponge in patients following hysterectomy, for pain control in hysterectomy. The patients were scheduled to receive a hysterectomy in the absence of uterine adenocarcinoma, cervical cancer, leiomyosarcoma or the suspicion of these cancers. Enrolled patients were to receive a total of three 5 cm×5 cm×0.5 cm bupivacaine-collagen sponges implanted at specified layers in the wound prior to wound closure. Each bupivacaine-collagen sponge was impregnated with 4 mg/cm³ of bupivacaine.

### Patient Selection Criteria

### Inclusion

Aged between 18 to 60 years
60-95 Kg in weight.
Able and willing to comply with pain relief regime outlined in the protocol

### Exclusion

Known hypersensitivity to amide local anaesthetics, NSAIDS, opioids and bovine collagen
Cardiac arrhythmias or AV conduction disorders
Concomitant use of other amide local anaesthetics
Concomitant use of anti-arrhythmics e.g. Amiodarone

### Concomitant Use of Propanolol

### Spinal Blockade

Concomitant use of strong/moderate CYP3A4 inhibitors or inducers e.g. macrolide antibiotics, grapefruit juice etc. Previous major surgery in last 6 months

### Hepatic Impairment

Any clinically significant unstable cardiac, neurological, immunological, renal or haematological disease or any other condition that in the opinion of the Investigator would interfere with the course of the study
Participation in a clinical trial using an Investigational
Product in the previous 6 months
Haemodynamically unstable at any point in the previous 4 weeks
Requirement for blood transfusion in the previous 3 months Haemoglobin below 10 g/dL

### Objectives

### Objectives

To investigate the potential analgesic effect of the bupivacaine-collagen sponge in the hysterectomy wound.
To investigate the morphine sparing effect of using bupivacaine-collagen sponge as part of the post-operative analgesia regimen.
To evaluate trends in numerical rating scales of pain intensity
   The study outcomes comprise:
Visual analogue pain rating scale (VAS)
Morphine sparing effect

### Study Duration

Screening assessments (informed consent, medical history, vital signs, 12-lead electrocardiogram, clinical biochemistry and haematology, urinalysis and demographics) took place between 1 and 14 days prior to administration of the bupivacaine-collagen sponge. Baseline (0 hours) procedures included allocation of study number, baseline pain scoring and implantation of the sponge. Follow-up procedures took place over 96 hours post implantation. A follow-up call was performed 8 days after implantation.

### Methodology

### Safety Analysis

Vital signs and clinical assessment for signs of systemic bupivacaine toxicity were repeated at 30 mins, 1, 1.5, 2, 3, 4, 5, 6, 9, 12 & 96 hours. A follow-up call was to be performed 8 days after surgery to check if the patient was suffering from any adverse event or is experiencing any problem with the wound. Concomitant medication (including doses of morphine and other pain medications) was collected from the screening until assessment 18.

TABLE 1
Safety Blood and Urinalysis Tests

| Haematology | Blood Chemistry | | Urinalysis |
|---|---|---|---|
| Haematocrit | Sodium | Total | pH |
| | | bilirubin | |
| Haemoglobin | Chloride | Direct | Protein |
| | | bilirubin | |
| RBC | Magnesium | ALT (SGPT) | Glucose |
| Platelet | Potassium | AST (SGOT) | Occult |
| | | | blood |
| WBC with | Calcium | Ferritin | |
| differential | | | |
| PT | Bicarbonate | Transferritin | |
| PTT | Glucose | GGT | |
| | Phosphorus | Iron | |
| | Uric Acid | C-reactive | |
| | | protein | |
| | Creatinine | Total | |
| | | Cholesterol | |
| | Blood Urea | Triglycerides | |
| | Nitrogen (BUN) | | |

### Efficacy Evaluations

Pain scoring using a visual analogue score (VAS) was used to assess the patient's experience of pain at 1, 1.5, 2, 3, 6, 9, 12, 18*, 24, 36, 48, 72 & 96 hour timepoints. Following surgery, patients were provided with PRN (per re nata, as needed) morphine via a PCA pump. Patient demand for morphine was recorded. The pain assessment at 18 hours was optional so that sleep is not disturbed.

### List of Abbreviations

### AE Adverse Events

AUCₗₐₛₜ Area under the plasma concentration-time curve from time zero to time t (time of last quantifiable plasma concentration)
AUC_{inf} Area under the plasma concentration-time curve from time zero to infinity
Cₘₐₓ Maximum plasma concentration
LOQ Limit of quantification

### PCA Patient Controlled Analgesia

PRN per re nata (as needed)
t_{lag} Lag-time
tₘₐₓ Time of maximum plasma concentration
t_{1/2} Terminal half life
λ_{Z} Terminal phase rate constant

### VAS Visual Analogue Scale

### Pain Control Medication (Supplemental to the Bupivacaine-Collagen Sponge)

At induction, patients received 50-75 μg fentanyl. Following catheterisation and prior to the commencement of surgery, they also received 100 mg diclofenac rectally. Intra-operatively, morphine was administered intravenously as required to maintain adequate levels of analgesia. Post-operatively, patients received 1 g paracetamol six hourly, 50 mg diclofenac every 8 hours, with a maximum of 3 doses in any 24 hours, and morphine PRN (per re nata, as needed) using a patient controlled analgesia pump.

### Concomitant Therapy

### Permitted Concomitant Therapies

In order to ensure conformity in the amount of analgesia received post-operatively, all patients received 1000 mg paracetamol at 6 hourly intervals and 50 mg diclofenac at 8 hourly intervals and morphine PRN using a PCA pump.

### Prohibited Concomitant Therapies

All analgesics were stopped for 24 hours prior to the study commencing. In addition, the following therapies (Table 2) were not administrated concomitantly with the implantation of the bupivacaine-collagen sponge.

TABLE 2
Prohibited Concomitant Therapies

| Strong or Moderate | |
|---|---|
| Inhibitors of the | Inducers of the CYP3A4 |
| CYP3A4 Pathway | Pathway |
| Grapefruit juice | troglitazone |
| Methadone | phenytoin |
| Itraconazole | rifampicin |
| Ketoconazole | carbamazepine |
| Fluconazole | phenobarbal |
| Clarithromycin | St. John's Wort (hypericin) |
| Erythromycin | |
| Nefazodone | |
| Fluoxetine | |
| Ritonavir | |
| Indinavir | |
| Nelfinavir | |
| Amprenavir | |
| Saquinavir | |

### Results

### Safety Analysis

Current Safety Data from Ongoing Clinical Study

| Pt | AE | | | | |
|---|---|---|---|---|---|
| No. | Description | Causality | Severity | Treatment | Outcome |
| 001 | Urine | Not | Moderate | (No | Resolved by |
| | infection | Available | | antibiotics) | Day 8 |
| 001 | Nausea on | Unlikely | Moderate | Odensatron | Resolved by |
| | days 1 and 2 | | | & cyclizine | Day 8 |
| 001 | Visual | Possible | Moderate | none | Resolved by |
| | disturbances | | | | Day 8 |
| 002 | Nausea | Not | Mild | (None) | Resolved |
| | | Available | | | |
| 003 | Increased | Not | (Details | (Details | Resolved by |
| | BP 1st few | Available | awaited) | awaited) | Day 2 |
| | hours | | | | |
| | considered | | | | |
| | normal for | | | | |
| | patient & | | | | |
| | not | | | | |
| | significant | | | | |
| 004 | Anemia HB | Not | Moderate | 2 units | HB 10 |
| | 11 to 8 | Available | | blood | |
| | | | | transfusion | |
| 004 | Urine | Not | Moderate | Augmentin | (Details |
| | infection | Available | | 625 mg TDS | awaited) |
| | | | | 5 days | |

### Efficacy Analysis

Efficacy of the bupivacaine-collagen sponge was determined by use of visual analogue scales and by the morphine sparing effect.

### Pain Scoring (Visual Analogue Pain Rating Scale)

Pain scoring using a visual analogue scale ranging from 0 (no pain) to 100 (worst imaginable pain). These were assessed from 1 hour onwards according to the assessment schedule. Individual pain scores were listed for each assessment time.

### Morphine Sparing

Patients were given 6 hourly doses of 1000 mg paracetamol, 8 hourly doses of 50 mg diclofenac and morphine PRN via patient controlled analgesia (PCA) pump. The doses of morphine demanded by each patient over the first 24 hours of the treatment period were recorded and compared to historical data in the literature. Total doses of morphine required over the treatment period were listed for each patient.

4 subjects have, to date, been enrolled in this study. The total morphine usage and pain scores (Visual Analog Scale of C to 10 mm where C is no pain and 100 is the worst imaginable pain) that have been reported by these 4 subjects are presented in Table 3.

TABLE 3
Summary of Morphine Usage and Pain Scores of
Subjects Enrolled in Ongoing Clinical Study

| | | Patient No. | | | |
|---|---|---|---|---|---|
| | | 001 | 002 | 003 | 004 |
| Age | | 35 | 45 | 45 | 48 |
| Date of enrollment | | 26-Jan-07 | 2-Feb-07 | 8-Feb-07 | 8-Feb-07 |
| (date of surgery) | | | | | |
| Total Amount of | | 28 | 7 | 0 | 1 |
| Morphine consumed | | | | | |
| (mgs) over first 24 | | | | | |
| hours post-op | | | | | |
| Pain scores | 1 h | 68 | 45 | 2 | 19 |
| (VAS) | 1.5 h | 49 | 34 | 7 | 19 |
| (mm) | 2 h | 43 | 56 | 13 | 30 |
| | 3 h | 43 | 65 | 13 | 24 |
| | 6 h | 16 | 5 | 0 | 10 |
| | 9 h | 4 | 2 | 0 | 12 |
| | 12 h | 4 | 0 | 6 | 6 |
| | 18 h | Not done | 0 | 4 | 6 |
| | 24 h | 14 | 3 | 2 | 4 |
| | 36 h | 24 | 2 | 3 | 0 |
| | 48 h | 2 | 0 | 0 | 0 |
| | 72 h | 1 | 9 | 0 | 0 |
| | 96 h | 0 | 6 | 0 | 0 |

| | | | | |
|---|---|---|---|---|
| Day of discharge | 5 | 4 | 5 | 5 |

Table 3 shows that the average postoperative morphine consumption was 9 mg over the first 24 hours post-op. In contrast, the average postoperative morphine consumption using bupivacaine infiltration in both deep and superficial layers of the wound after abdominal hysterectomy was 54 mg (Klein et al, 2000) and 44 mg (Ng et al, 2002) and narcotic consumption during the first 24 hours varied from 35 to 62 mg (Gupta, 2004).

The invention is not limited to the embodiments described and exemplified herein, which may be modified and amended without departing from the scope of the present invention. While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims.

### REFERENCES

Gupta A, Perniola A, Axelsson K, Thorn S E, Crafpprd K, and Rawal N (2004). Postoperative pain after abdominal hysterectomy: a double-blind comparison between placebo and local anesthetic infused intraperitoneally. Anesth Analg 99:1173-1179.
Leung C C, Chan Y M, Ngai S W, et al (2000). Effect of pre-incision skin infiltration on post-hysterectomy pain—a double-blind randomized controlled trial. Anaesth Intensive Care 2000; 28:510-516.
Ng A, Swami A, Smith G, et al (2002). The analgesic effects of intraperitoneal and incisional bupivacaine with epinephrine after total abdominal hysterectomy. Anesth Analg 2002; 95:158-162.
Sinclair et al, 1996 Postoperative pain relief by topical lidocaine in the surgical wound of hysterectomized patients; Acta Anaesthesiologica Scandinavica 40: 589-594 Hannibal et al, 1996 Preoperative wound infiltration with bupivacaine reduces early and late opioid requirements after hysterectomy. British Journal of Anaethesiology 83: 376-381
Cobby et al, 1997 Wound infiltration with local anaesthetic after abdominal hysterectomy British Journal of Anaethesiology: 78: 431-432
Victory et al, 1995 Effect of preincision versus postincision infiltration with bupivacaine on postoperative pain Journal of Clinical Anesthesia 7:192-196
Bartholdy et al, 1994 Preoperative infiltration of the surgical area enhances postoperative analgesia of a combined low-dose epidural bipivacaine and morphine regimen after upper abdominal surgery. Acta Anaesthesiologica Scandinavica 38: 262-265
Møiniche et al, 1998 A qualitative systemic review of incisional local anaesthesia for postoperative pain relief after abdominal operations; British Journal of Anaesthesia; 81: 377-383
Klein et al, 2000 Infiltration of the abdominal wall with local anaesthetic after total abdominal hysterectomy with no opioid-sparing effect; British Journal of Anaesthesia 84 (2): 248-9

What is claimed is:

1. A drug delivery device for providing local analgesia, local anesthesia or nerve blockade at a site in a human or animal in need thereof, the device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration.

2. The drug delivery device of claim 1, wherein the at least one drug substance is present in an amount sufficient to provide a duration of local analgesia, local anesthesia or nerve blockade which lasts for at least about two days after administration.

3. The drug delivery device of claim 1, wherein the at least one drug substance is present in an amount sufficient to provide a duration of local analgesia, local anesthesia or nerve blockade which lasts for at least about three days after administration.

4. The drug delivery device of claim 1, wherein the at least one drug substance is an amino amide anesthetic selected from the group comprising Bupivacaine, Levobupivacaine, Lidocaine, Mepivacaine, Prilocaine, Ropivacaine, Articaine, Trimecaine and their salts and prodrugs.

5. The drug delivery device of claim 1, wherein the at least one drug substance is an amino amide anesthetic selected from bupivacaine and salts and prodrugs thereof.

6. The drug delivery device of claim 1, wherein the fibrillar collagen matrix is a Type 1 collagen matrix.

7. The drug delivery device of claim 1, wherein the fibrillar collagen matrix is a Type 1 collagen matrix and the at least one drug substance is an amino amide anesthetic selected from bupivacaine and salts and prodrugs thereof.

8. The drug delivery device of claim 7, wherein the drug delivery device comprises a plurality of collagen sponges, each collagen sponge containing about 3.6 to about 8.0 mg/cm³ type I collagen and about 2.0 to about 6.0 mg/cm³ bupivacaine hydrochloride.

9. The drug delivery device of claim 7, wherein the drug delivery device comprises a plurality of collagen sponges, each collagen sponge containing about 5.6 mg/cm³ type I collagen and about 4.0 mg/cm³ bupivacaine hydrochloride.

10. A method for providing local analgesia, local anesthesia or nerve blockade in a human or animal in need thereof, the method comprising administering at a site in a human or animal in need thereof a drug delivery device comprising a fibrillar collagen matrix; and at least one drug substance selected from the group consisting of amino amide anesthetics, amino ester anesthetics and mixtures thereof, the at least one drug substance being substantially homogeneously dispersed in the collagen matrix, and the at least one drug substance being present in an amount sufficient to provide a duration of local analgesia, local anesthesia or nerve blockade which lasts for at least about one day after administration.

11. The method of claim 10, wherein the at least one drug substance is present in an amount sufficient to provide a duration of local analgesia, local anesthesia or nerve blockade which lasts for at least about two days after administration.

12. The method of claim 10, wherein the at least one drug substance is present in an amount sufficient to provide a duration of local analgesia, local anesthesia or nerve blockade which lasts for at least about three days after administration.

13. The method of claim 10, wherein the at least one drug substance is an amino amide anesthetic selected from the group comprising Bupivacaine, Levobupivacaine, Lidocaine, Mepivacaine, Prilocaine, Ropivacaine, Articaine, Trimecaine and their salts and prodrugs.

14. The method of claim 10, wherein the at least one drug substance is an amino amide anesthetic selected from bupivacaine and salts and prodrugs thereof.

15. The method of claim 10, wherein the fibrillar collagen matrix is a Type 1 collagen matrix.

16. The method of claim 10, wherein the fibrillar collagen matrix is a Type 1 collagen matrix and the at least one drug substance is an amino amide anesthetic selected from bupivacaine and salts and prodrugs thereof.

17. The method of claim 16, wherein the drug delivery device comprises a plurality of collagen sponges, each collagen sponge containing about 3.6 to about 8.0 mg/cm³ type I collagen and about 2.0 to about 6.0 mg/cm³ bupivacaine hydrochloride.

18. The method of claim 16, wherein the drug delivery device comprises a plurality of collagen sponges, each collagen sponge containing about 5.6 mg/cm³ type I collagen and about 4.0 mg/cm³ bupivacaine hydrochloride.

19. The method of claim 10, wherein the method is for providing local analgesia, local anesthesia or nerve blockade in a human following laparotomy.

20. The method of claim 10, wherein the method is for providing local analgesia, local anesthesia or nerve blockade in a human following hysterectomy.

21. The method of claim 17, wherein the method is for providing local analgesia, local anesthesia or nerve blockade in a human following hysterectomy.

22. The method of claim 18, wherein the method is for providing local analgesia, local anesthesia or nerve blockade in a human following hysterectomy.

23. The method of claim 10, wherein the drug delivery device comprises a plurality of collagen sponges and wherein one sponge is divided between areas in the surgical vault, one sponge is divided and placed across the incision in the peritoneum and one sponge is divided and placed between the sheath and skin around the incision.
